(19) United States
(12) Patent Application Publication
(10) Pub. No.: US 2020/0188030 A1
(43) Pub. Date: Jun. 18, 2020

(54) Augmented Reality-Based Navigation for Use in Surgical and Non-Surgical Procedures

(71) Applicant: Duke University, Durham, NC (US)

(72) Inventors: Regis Kopper, Durham, NC (US); David J. Zielinski, Durham, NC (US); Andrew Cutler, Durham, NC (US); Nandan Lad, Durham, NC (US); Patrick Codd, Durham, NC (US); Shervin Rahimpour, Durham, NC (US)

(21) Appl. No.: 16/484,444

(22) PCT Filed: Feb. 8, 2018
(86) PCT No.: PCT/US2018/017381

§ 371 Date: Aug. 7, 2019

Related U.S. Application Data
(60) Provisional application No. 62/456,205, filed on Feb. 8, 2017.

(51) Int. Cl. A61B 34/20; A61B 5/00; A61B 5/06; A61B 6/03; A61B 6/00; A61B 5/11; G02B 27/01; G16H 30/40; G16H 20/40

(52) U.S. Cl. CPC A61B 34/20; G02B 2027/0174; A61B 5/7475; A61B 5/062; A61B 5/7455; A61B 5/7405; A61B 5/749; A61B 6/032; A61B 6/501; A61B 5/1114; A61B 5/064; G02B 27/0172; G02B 27/0179; G16H 30/40; G16H 20/40; A61B 2562/0204; A61B 2562/0219; A61B 2562/0223; A61B 2034/2048; A61B 5/745

(57) ABSTRACT

The present disclosure provides a wearable computing device including (i) one or more sensors, (ii) a partially or fully transparent display, and (iii) a control system. The control system is configured to receive data indicative of a three-dimensional model of a body part. The control system is further configured to receive sensor data from the one or more sensors. Based on the sensor data, the control system is further configured to determine a relative position of a physical body part of a physical body with respect to the wearable computing device. Based on the determined relative position, the control system is configured to cause the display to provide a hologram corresponding to at least a portion of the three-dimensional model of the body part such that the hologram appears overlaid onto at least a portion of the physical body part when viewed through the display.

### RELATED APPLICATIONS

This application claims the benefit of priority to U.S. Provisional Application No. 62/456,205 entitled “Augmented Reality-Based Navigation for Use in Surgical and Non-Surgical Procedures,” filed on Feb. 8, 2017, the contents of which is hereby incorporated by reference in its entirety.

### BACKGROUND

Unless otherwise indicated herein, the materials described in this section are not prior art to the claims in this application and are not admitted to be prior art by inclusion in this section.

The trend toward miniaturization of computing hardware, peripherals, as well as of sensors, detectors, and image and audio processors, among other technologies, has helped open up a field sometimes referred to as “wearable computing.” In the area of image and visual processing and production, in particular, it has become possible to consider wearable displays that place a graphic display close enough to a wearer's (or user's) eye(s) such that the displayed image appears as a normal-sized image, such as might be displayed on a traditional image display device. The relevant technology may be referred to as “near-eye displays.”

Wearable computing devices with near-eye displays may also be referred to as “head-mountable displays” (HMDs), “head-mounted displays,” “head-mounted devices,” or “head-mountable devices.” A head-mountable display places a graphic display or displays close to one or both eyes of a wearer. To generate the images on a display, a computer processing system may be used. Such displays may occupy a wearer's entire field of view, or only occupy part of wearer's field of view. Further, head-mounted displays may vary in size, taking a smaller form such as a glasses-style display or a larger form such as a helmet, for example.

Emerging and anticipated uses of wearable displays include applications in which users interact in real time with an augmented reality (which may also be referred to as mixed reality) environment and/or with a virtual reality environment. Such applications can be mission-critical or safety-critical, such as in medical procedures. In particular, certain surgical or non-surgical medical procedures require a medical professional to interact with body parts that are hidden from view. Such procedures may be challenging to perform since the medical professional cannot see the particular body part upon which they are performing the procedure. As such, there is a need for augmented reality-based navigation in order to perform surgical and non-surgical procedures.

### SUMMARY

In a first aspect, a wearable computing device is provided including (i) one or more sensors, (ii) a partially or fully transparent display, and (iii) a control system. The control system is configured to receive data indicative of a three-dimensional model of a body part. The control system is further configured to receive sensor data from the one or more sensors. Based on the sensor data, the control system is further configured to determine a relative position of a physical body part of a physical body with respect to the wearable computing device. Based on the determined relative position, the control system is configured to cause the display to provide a hologram corresponding to at least a portion of the three-dimensional model of the body part such that the hologram appears overlaid onto at least a portion of the physical body part when viewed through the display.

In a second aspect, a computer-implemented method operable by a wearable computing device is provided. The method includes receiving data indicative of a three-dimensional model of a body part. The method further includes receiving sensor data from one or more sensors of the wearable computing device. The method further includes, based on the sensor data, determining a relative position of a physical body part of a physical body with respect to the wearable computing device. The method further includes, based on the determined relative position, causing a partially or fully transparent display of the wearable computing device to provide a hologram corresponding to at least a portion of the three-dimensional model of the body part such that the hologram appears overlaid onto at least a portion of the physical body part when viewed through the display.

In a third aspect, a non-transitory computer-readable medium having stored thereon instructions that, when executed by one or more processors of a wearable computing device, cause the head-mountable device to perform functions. The functions include receiving data indicative of a three-dimensional model of a body part. The functions further include receiving sensor data from one or more sensors of the wearable computing device. The functions further include, based on the sensor data, determining a relative position of a physical body part of a physical body with respect to the wearable computing device. The functions further include, based on the determined relative position, causing a partially or fully transparent display of the wearable computing device to provide a hologram corresponding to at least a portion of the three-dimensional model of the body part such that the hologram appears overlaid onto at least a portion of the physical body part when viewed through the display.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a wearable computing device according to an example embodiment.

FIG. 2 is a simplified block diagram of a computing device according to an example embodiment.

FIG. 3 a simplified flow chart illustrating a method, according to an example embodiment.

FIG. 4A illustrates a medical professional performing a medical procedure, according to an example embodiment.

FIG. 4B illustrates a medical professional with a wearable computing device performing a medical procedure, according to an example embodiment.

FIG. 4C illustrates the medical professional of FIG. 4B further performing the medical procedure, according to an example embodiment.

FIG. 4D illustrates a first person view of the medical professional of FIG. 4B performing the medical procedure of FIG. 4B, according to an example embodiment.

FIG. 4E illustrates a first person view of the medical professional of FIG. 4B performing the procedure of FIG. 4C, according to an example embodiment.

FIG. 5 depicts a computer-readable medium configured according to an example embodiment.

### DETAILED DESCRIPTION

Example methods and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features. In the following detailed description, reference is made to the accompanying figures, which form a part thereof. In the figures, similar symbols typically identify similar components, unless context dictates otherwise. Other embodiments may be utilized, and other changes may be made, without departing from the scope of the subject matter presented herein.

The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

As used herein, with respect to measurements, “about” means +/−5%.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

In the following description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts will be described in conjunction with specific examples, it will be understood that these examples are not intended to be limiting.

### A. OVERVIEW

Example embodiments may generally relate to a wearable computing device that utilizes an augmented reality-based display to assist in surgical and non-surgical procedures.

As discussed above, certain surgical or non-surgical medical procedures may involve a medical professional interacting with body parts that are hidden from view. Such procedures may be challenging to perform since the medical professional cannot directly view the particular body part upon which they are performing the procedure. Example embodiments described herein provide augmented reality-based navigation in order to help perform such procedures.

In particular, an example wearable computing device may include (i) one or more sensors, (ii) a partially or fully transparent display, and (iii) a control system. In one example, the wearable computing device takes the form of or includes a head-mountable device. The control system may be configured to receive data indicative of a three-dimensional model of a body part. For example, the data indicative of the three-dimensional model of the body part that is received by the wearable computing device may be based on a computed tomography (CT) scan of a brain. As described elsewhere herein, the three-dimensional model of the body part received by the wearable computing device could be based on other 2D and/or 3D imaging techniques, such as magnetic resonance imaging (MRI), ultrasound, x-ray, etc.

The control system may be further configured to receive sensor data from the one or more sensors. Based on the sensor data, the control system may be further configured to determine a relative position of a physical body part of a physical body with respect to the wearable computing device. Based on the determined relative position, the control system may be configured to cause the display to provide a hologram corresponding to at least a portion of the three-dimensional model of the body part such that the hologram appears overlaid onto at least a portion of the physical body part when viewed through the display. A wearer of the wearable computing device can then use the hologram as a proxy for the actual location of the body part, and can utilize one or more medical devices to perform the procedure on the body part.

It should be understood that the above examples of the method are provided for illustrative purposes, and should not be construed as limiting.

### B. EXAMPLE WEARABLE COMPUTING DEVICES

Systems and devices in which example embodiments may be implemented will now be described in greater detail. In one embodiment, an example computing device may take the form of a wearable computing device (also referred to as a wearable computer). In an example embodiment, a wearable computing device takes the form of or includes a head-mountable device (HMD).

An HMD may generally be any display device that is capable of being worn on the head and places a display in front of one or both eyes of the wearer. An HMD may take various forms such as a helmet or eyeglasses. As such, references to “eyeglasses” or a “glasses-style” HMD should be understood to refer to an HMD that has a glasses-like frame so that it can be worn on the head. Further, example embodiments may be implemented by or in association with an HMD with a single display or with two displays, which may be referred to as a “monocular” HMD or a “binocular” HMD, respectively.

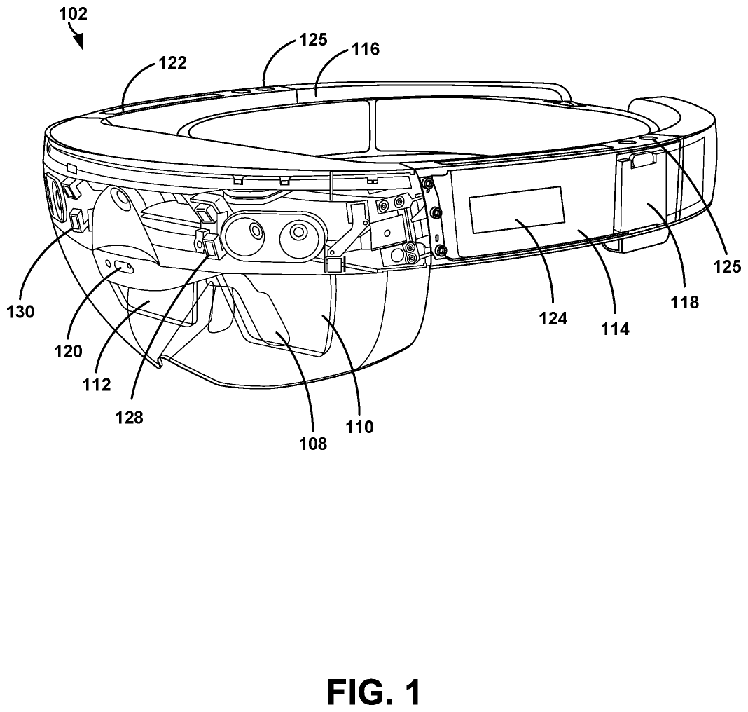

FIG. 1 illustrates a wearable computing device 102 according to an example embodiment. In FIG. 1, the wearable computing device 102 takes the form of a head-mountable device (HMD) (which may also be referred to as a head-mounted display). As illustrated, the wearable computing device 102 could take the form of a Microsoft Hololens or another type of augmented reality device. It should be understood, however, that example systems and devices may take the form of or be implemented within or in association with other types of devices, without departing from the scope of the invention. As illustrated in FIG. 1, the wearable computing device 102 includes frame elements including a center frame support 108, lens elements 110, 112 thereby defining a display, and extending side-arms 114, 116. The center frame support 108 and the extending side-arms 114, 116 are configured to secure the wearable computing device 102 to a user's face via a user's nose and head, respectively.

Each of the center frame support 108 and the extending side-arms 114, 116 may be formed of a solid structure of plastic and/or metal, or may be formed of a hollow structure of similar material so as to allow wiring and component interconnects to be internally routed through the wearable computing device 102. Other materials may be possible as well.

One or more of each of the lens elements 110, 112 may be formed of any material that can suitably display a projected image or graphic. Each of the lens elements 110, 112 may also be sufficiently transparent to allow a user to see through the lens element. Combining these two features of the lens elements 110, 112 together comprise a display that may facilitate an augmented reality or heads-up display where the projected image or graphic is superimposed over a real-world view as perceived by the user through the lens elements 110, 112.

The extending side-arms 114, 116 may each be projections that extend away from the lens elements 110, 112, respectively, and may be positioned behind a user's ears to secure the wearable computing device 102 to the user. The extending side-arms 114, 116 may further secure the wearable computing device 102 to the user by extending around a rear portion of the user's head. Additionally or alternatively, for example, the wearable computing device 102 may connect to or be affixed within a head-mounted helmet structure. Other configurations for a wearable computing device are also possible.

The wearable computing device 102 may also include an on-board computing system 118, an image capture device 120, a sensor 122, and a finger-operable touch pad 124. The on-board computing system 118 is shown to be positioned on the extending side-arm 114 of the wearable computing device 102; however, the on-board computing system 118 may be provided on other parts of the wearable computing device 102 or may be positioned remote from the wearable computing device 102 (e.g., the on-board computing system 118 could be wire- or wirelessly-connected to the wearable computing device 102). The on-board computing system 118 may include a processor and memory, for example. The on-board computing system 118 may be configured to receive and analyze data from the image capture device 120 and the finger-operable touch pad 124 (and possibly from other sensory devices, user interfaces, or both) and generate images for output by the lens elements 110 and 112.

The image capture device 120 may be, for example, a camera that is configured to capture still images and/or to capture video. In the illustrated configuration, image capture device 120 is positioned above the lens elements 110, 112 of the wearable computing device 102; however, the image capture device 120 may be provided on other parts of the wearable computing device 102. The image capture device 120 may be configured to capture images at various resolutions or at different frame rates. Many image capture devices with a small form-factor, such as the cameras used in mobile phones or webcams, for example, may be incorporated into an example of the wearable computing device 102.

Further, although FIG. 1 illustrates one image capture device 120, more image capture device may be used, and each may be configured to capture the same view, or to capture different views. For example, the image capture device 120 may be forward facing to capture at least a portion of the real-world view perceived by the user. This forward facing image captured by the image capture device 120 may then be used to generate an augmented reality where computer generated images appear to interact with or overlay the real-world view perceived by the user. In some embodiments, the computer generated images may appear to be registered or otherwise aligned to real-world objects or features, as described herein.

The sensor 122 is shown on the extending side-arm 116 of the wearable computing device 102; however, the sensor 122 may be positioned on other parts of the wearable computing device 102. For illustrative purposes, only one sensor 122 is shown. However, in an example embodiment, the wearable computing device 102 may include multiple sensors. For example, a wearable computing device 102 may include sensors 122 such as one or more gyroscopes, one or more accelerometers, one or more magnetometers, one or more light sensors, one or more infrared sensors, and/or one or more microphones. Other sensing devices may be included in addition or in the alternative to the sensors that are specifically identified herein.

The finger-operable touch pad 124 is shown on the extending side-arm 114 of the wearable computing device 102. However, the finger-operable touch pad 124 may be positioned on other parts of the wearable computing device 102. Also, more than one finger-operable touch pad may be present on the wearable computing device 102. The finger-operable touch pad 124 may be used by a user to input commands. The finger-operable touch pad 124 may sense at least one of a pressure, position and/or a movement of one or more fingers via capacitive sensing, resistance sensing, or a surface acoustic wave process, among other possibilities. The finger-operable touch pad 124 may be capable of sensing movement of one or more fingers simultaneously, in addition to sensing movement in a direction parallel or planar to the pad surface, in a direction normal to the pad surface, or both, and may also be capable of sensing a level of pressure applied to the touch pad surface. In some embodiments, the finger-operable touch pad 124 may be formed of one or more translucent or transparent insulating layers and one or more translucent or transparent conducting layers. Edges of the finger-operable touch pad 124 may be formed to have a raised, indented, or roughened surface, so as to provide tactile feedback to a user when the user's finger reaches the edge, or other area, of the finger-operable touch pad 124. If more than one finger-operable touch pad is present, each finger-operable touch pad may be operated independently, and may provide a different function.

In a further aspect, wearable computing device 102 may be configured to receive user input in various ways, in addition or in the alternative to user input received via finger-operable touch pad 124. For example, on-board computing system 118 may implement a speech-to-text process and utilize a syntax that maps certain spoken commands to certain actions. In addition, the wearable computing device 102 may include one or more microphones via which a wearer's speech may be captured. Configured as such, the wearable computing device 102 may be operable to detect spoken commands and carry out various computing functions that correspond to the spoken commands.

As another example, the wearable computing device 102 may interpret certain head-movements as user input. For example, when the wearable computing device 102 is worn, wearable computing device 102 may use one or more gyroscopes and/or one or more accelerometers to detect head movement. The wearable computing device 102 may then interpret certain head-movements as being user input, such as nodding, or looking up, down, left, or right. A wearable computing device 102 could also pan or scroll through graphics in a display according to movement. Other types of actions may also be mapped to head movement.

As yet another example, wearable computing device 102 may interpret certain gestures (e.g., by a wearer's hand or hands) as user input. For example, wearable computing device 102 may capture hand movements by analyzing image data from image capture device 120, and initiate actions that are defined as corresponding to certain hand movements.

As a further example, wearable computing device 102 may interpret eye movement as user input. In particular, wearable computing device 102 may include one or more inward-facing image capture devices and/or one or more other inward-facing sensors that may be used to track eye movements, authenticate a user, and/or determine the direction of a user's gaze. As such, certain eye movements may be mapped to certain actions. For example, certain actions may be defined as corresponding to movement of the eye in a certain direction, a blink, and/or a wink, among other possibilities.

Wearable computing device 102 also includes a speaker 125 for generating audio output. In one example, the speaker could be in the form of a bone conduction speaker, also referred to as a bone conduction transducer (BCT). Speaker 125 may be, for example, a vibration transducer or an electroacoustic transducer that produces sound in response to an electrical audio signal input. The frame of wearable computing device 102 may be designed such that when a user wears wearable computing device 102, the speaker 125 contacts the wearer. Alternatively, speaker 125 may be embedded within the frame of wearable computing device 102 and positioned such that, when the wearable computing device 102 is worn, speaker 125 vibrates a portion of the frame that contacts the wearer. In either case, wearable computing device 102 may be configured to send an audio signal to speaker 125, so that vibration of the speaker may be directly or indirectly transferred to the bone structure of the wearer. When the vibrations travel through the bone structure to the bones in the middle ear of the wearer, the wearer can interpret the vibrations provided by BCT 125 as sounds.

Various types of bone-conduction transducers (BCTs) may be implemented, depending upon the particular implementation. Generally, any component that is arranged to vibrate the wearable computing device 102 may be incorporated as a vibration transducer. Yet further it should be understood that a wearable computing device 102 may include a single speaker 125 or multiple speakers. In addition, the location(s) of speaker(s) on the wearable computing device 102 may vary, depending upon the implementation. For example, a speaker may be located proximate to a wearer's temple (as shown), behind the wearer's ear, proximate to the wearer's nose, and/or at any other location where the speaker 125 can vibrate the wearer's bone structure.

As shown in FIG. 1, the lens elements 110, 112 may act as display elements. The wearable computing device 102 may include a first projector 128 configured to project a display onto an inside surface of the lens element 110. Additionally or alternatively, a second projector 130 may be configured to project a display onto an inside surface of the lens element 112. The lens elements 110, 112 may act as a combiner in a light projection system and may include a coating that reflects the light projected onto them from the projectors 128, 130. In some embodiments, a reflective coating may not be used (e.g., when the projectors 128, 130 are scanning laser devices).

In alternative embodiments, other types of display elements may also be used. For example, the lens elements 110, 112 themselves may include: a transparent or semi-transparent matrix display, such as an electroluminescent display or a liquid crystal display, one or more waveguides for delivering an image to the user's eyes, or other optical elements capable of delivering an in focus near-to-eye image to the user. A corresponding display driver may be disposed within the wearable computing device 102 for driving such a matrix display. Alternatively or additionally, a laser or LED source and scanning system could be used to draw a raster display directly onto the retina of one or more of the user's eyes. Other possibilities exist as well.

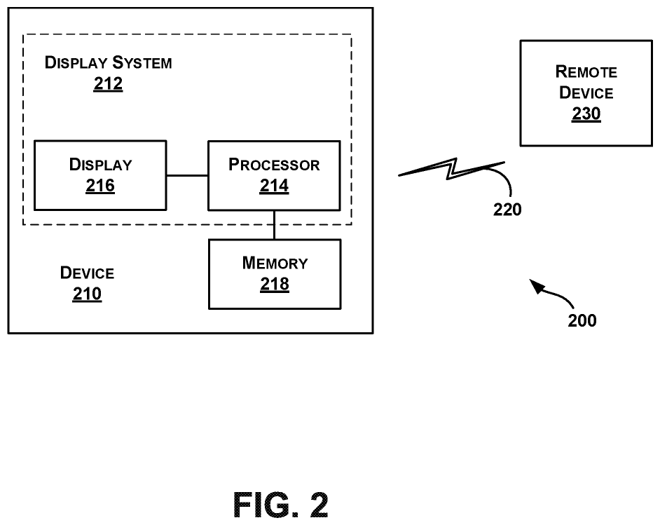

FIG. 2 is a simplified block diagram a computing device 210 according to an example embodiment. In an example embodiment, computing device 210 communicates using a communication link 220 (e.g., a wired or wireless connection) to a remote device 230. The computing device 210 may be any type of device that can receive data and display information corresponding to or associated with the data. For example, the computing device 210 may take the form of or include a head-mountable display, such as the wearable computing device 102 that is described with reference to FIG. 1.

The computing device 210 may include a processor 214 and a display 216. The display 216 may be, for example, an optical see-through display, an optical see-around display, or a video see-through display. The processor 214 may receive data from the remote device 230, and configure the data for display on the display 216. The processor 214 may be any type of processor, such as a micro-processor or a digital signal processor, for example.

The computing device 210 may further include on-board data storage, such as memory 218 coupled to the processor 214. The memory 218 may store software that can be accessed and executed by the processor 214, for example.

The remote device 230 may be any type of computing device or transmitter including a laptop computer, a mobile telephone, head-mountable display, tablet computing device, etc., that is configured to transmit data to the computing device 210. The remote device 230 and the computing device 210 may contain hardware to enable the communication link 220, such as processors, transmitters, receivers, antennas, etc.

Further, remote device 230 may take the form of or be implemented in a computing system that is in communication with and configured to perform functions on behalf of client device, such as computing device 210. Such a remote device 230 may receive data from another computing device 210 (e.g., a wearable computing device 102 or a mobile phone), perform certain processing functions on behalf of the computing device 210, and then send the resulting data back to computing device 210. This functionality may be referred to as “cloud” computing.

In FIG. 2, the communication link 220 is illustrated as a wireless connection; however, wired connections may also be used. For example, the communication link 220 may be a wired serial bus such as a universal serial bus or a parallel bus. A wired connection may be a proprietary connection as well. The communication link 220 may also be a wireless connection using, e.g., BLUETOOTH radio technology, BLUETOOTH LOW ENERGY (BLE), communication protocols described in IEEE 802.11 (including any IEEE 802.11 revisions), Cellular technology (such as GSM, CDMA, UMNTS, EV-DO, WiMAX, or LTE), or ZIGBEE technology, among other possibilities. The remote device 230 may be accessible via the Internet.

### C. EXAMPLES OF METHODS

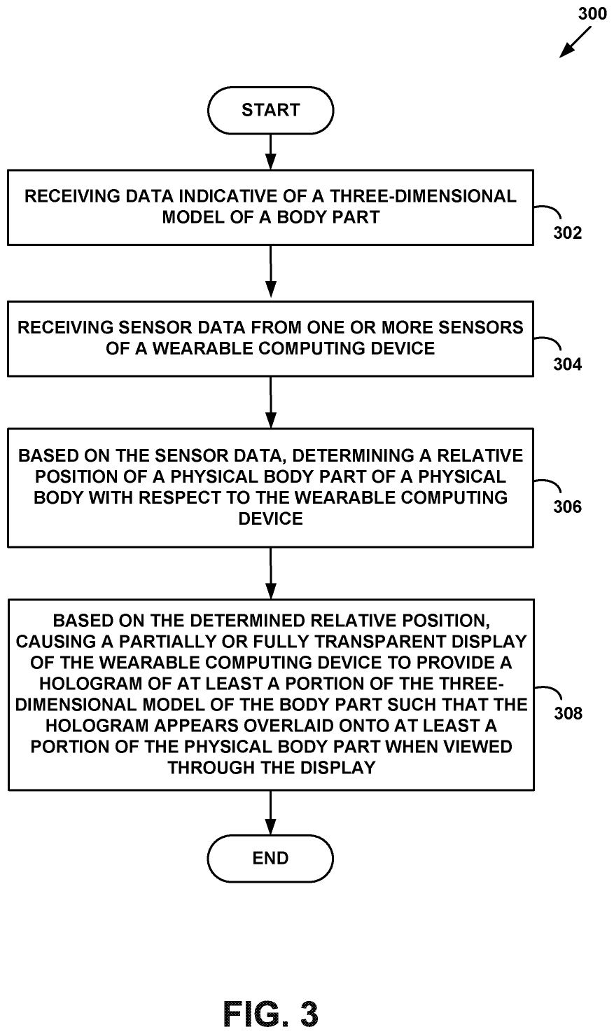

FIG. 3 is a simplified flow chart illustrating method 300. Although the blocks in FIG. 3 are illustrated in a sequential order, these blocks may also be performed in parallel, and/or in a different order than those described herein. Also, the various blocks may be combined into fewer blocks, divided into additional blocks, and/or removed based upon the desired implementation.

Further, while the methods described herein are described by way of example as being carried out by a wearable computing device, it should be understood that an exemplary method or a portion thereof may be carried out by another entity or combination of entities, without departing from the scope of the invention.

In addition, the flowchart of FIG. 3 shows functionality and operation of one possible implementation of present embodiments. In this regard, each block may represent a module, a segment, or a portion of program code, which includes one or more instructions executable by a processor for implementing specific logical functions or steps in the process. The program code may be stored on any type of computer-readable medium, for example, such as a storage device including a disk or hard drive. The computer-readable medium may include non-transitory computer-readable medium, for example, such as computer-readable media that stores data for short periods of time like register memory, processor cache and Random Access Memory (RAM). The computer-readable medium may also include non-transitory media, such as secondary or persistent long term storage, like read only memory (ROM), optical or magnetic disks, compact-disc read only memory (CD-ROM), for example. The computer-readable media may also be any other volatile or non-volatile storage systems. The computer-readable medium may be considered a computer-readable storage medium, for example, or a tangible storage device.

Referring again to FIG. 3, method 300 involves a steps performed by a computing device. In one example, the computing device may be implemented as part of a wearable device, such as an HMD or component thereof (e.g., wearable computing device 102 described above). Such a wearable computing device may include (i) one or more sensors, (ii) a partially or fully transparent display, and (iii) a control system, as discussed above in relation to FIGS. 1 and 2. In such an example, the control system may perform the method steps 302-308. In another example, the computing device may be a separate entity in wireless communication with the wearable computing device. As such, one or more of the method steps 302-308 may be performed by a computing device other than the wearable computing device.

At block 302, the method 300 includes receiving data indicative of a three-dimensional model of a body part. In one example, the data indicative of the three-dimensional model of the body part is based on a computed tomography (CT) scan of a brain. In another example, the data indicative of the three-dimensional model of the body part is based on a CT scan of another body part, such as another organ other than the brain or a bone, as examples. In another example, the data indicative of the three-dimensional model of the body part is based on a magnetic resonance imaging (MRI) scan of a body part, an X-ray of a body part, or some other medical imaging system of a body part. In yet another example, the data indicative of the three-dimensional model of the body part comprises a real-time three-dimensional model of the physical body part. In such an example, the data indicative of the three-dimensional model of the body part is received from an ultrasound device. Other examples are possible as well. The data indicative of the three-dimensional model of the body part may be received by the wearable computing device via a wired or wireless connection with another computing device. The data indicative of the three-dimensional model of the body part may be received in a variety of file types and data formats, such as pseudo-3D or true-3D file/format types (e.g., 3D vector formats like .dwg, .dxf, VRML, X3D, etc.). Other file/format types are possible as well.

At block 304, the method 300 includes receiving sensor data from one or more sensors of the wearable computing device. The one or more sensors of the wearable computing device may include one or more proximity sensors, one or more touch interfaces, one or more microphones, one or more accelerometers, one or more gyroscopes, or one or more magnetometers. Other sensing devices may be included in addition or in the alternative to the sensors that are specifically identified herein. The one or more sensors may be integral to or separate from the wearable computing device. For example, the one or more sensors may be integrated on a wearable computing device, or may be remote to the wearable computing device (such as biometric sensors placed on other portions of the body or in communication with the body). The one or more sensors may also be provided on a computing device remote from the wearable computing device (such as a remote device such as a smartphone having location tracking and internet capabilities). The one or more sensors are configured to capture various data of the wearer of the wearable computing device and send the sensor data to the computing device for further analysis.

The method 300 continues at block 306 with, based on the sensor data, determining a relative position of a physical body part of a physical body with respect to the wearable computing device. The sensor data may include information about a location of the wearable computing device, a location of the physical body part of the physical body, or both. In one example, the sensor data determining the relative position of the physical body part of the physical body with respect to the wearable computing device is further based on three or more markers positioned on the physical body. In such an example, the three or more markers may be positioned on the physical body (e.g., via a temporary adhesive on one side of each of the three or more markers). The physical body part of the physical body may then be captured using an image capture system, such as a CT scan. The CT scan captures both the physical body part, and the three or more markers positioned on the physical body. The respective positions of, and/or the respective distances between, the three or more markers and the physical body part may then be used to determine the relative position of the physical body part of the physical body with respect to the wearable computing device.

The method 300 continues at block 308 with, based on the determined relative position, causing a partially or fully transparent display of the wearable computing device to provide a hologram corresponding to at least a portion of the three-dimensional model of the body part such that the hologram appears overlaid onto at least a portion of the physical body part when viewed through the display. Using the example of the three or more markers described above, the hologram corresponding to at least a portion of the three-dimensional model of the body part may be overlaid onto at least a portion of the physical body part by aligning the three or more markers in the CT scan with the three or more markers positioned on the body part. In another example, the hologram corresponding to at least a portion of the three-dimensional model of the body part may be overlaid onto at least a portion of the physical body part by using facial recognition and aligning facial features from the CT scan with facial features of the body part. As used herein, the physical body part may comprise a training model or an animal body part and need not be a human body part.

In another example, the hologram corresponding to at least a portion of the three-dimensional model of the body part may be overlaid onto at least a portion of the physical body part by an external tracking system and a calibration image positioned adjacent to the physical body part. The calibration image may be any image that can be seen by visual cameras that are used to calibrate the wearable computing device. The calibration image could include a QR code, a marker, a bar code, a predetermined shape and/or pattern, or image target, as non-limiting examples. The data from the external tracking system may be sent wirelessly to the wearable computing device. The wearable computing device may then use this data to align the hologram with the body part. Other ways to align the hologram with the body part are possible as well.

In one example, the wearable computing device further includes an audio input device. In such an example, the method 300 further includes (i) receiving, from the audio input device, one or more verbal inputs, and (ii) adjusting a location of the hologram based on the one or more verbal inputs. For example, the wearer may say “move” to move the hologram to a specific location, and “fix” to anchor the location of the hologram relative to the physical world and prevent it from moving as the wearer walks around. If the wearer wants to delete the anchor, the user can say “delete anchor”. Further, the wearer of the wearable computing device may show/hide the hologram corresponding to at least a portion of the three-dimensional model of the body part via an audio input. It will be understood that other verbal inputs (e.g., different trigger words or trigger phrases) are possible and contemplated herein.

In another example, the wearable computing device may interpret certain gestures (e.g., by a wearer's hand or hands) as user input. For example, the wearable computing device may capture hand movements by analyzing image data from image capture device the wearable computing device, and initiate actions that are defined as corresponding to certain hand movements. Such gestures may include moving and rotating the hologram relative to the physical world. These gestures may be used to position the hologram corresponding to at least a portion of the three-dimensional model of the body part overlaid onto at least a portion of the physical body part.

In another example, the method 300 further includes (i) determining a relative position of a medical device with respect to the wearable computing device, and (ii) based on the determined relative position of the medical device, causing the display to provide a medical device hologram corresponding to at least a portion of the medical device that is positioned in the physical body. The relative position of the medical device may be determined in number of ways. In one example, the medical device may include a tracking sensor that the wearable computing device can detect to determine the location of the medical device relative to the wearable computing device. In another example, a plurality of markers may be positioned on the medical device that the wearable computing device can detect to determine the location of the medical device relative to the wearable computing device. In yet another example, the wearable computing device may store or have access to holographic models of a plurality of medical devices. In such an example, the wearer then selects via the wearable computing device the model of the medical device that matches the physical one they are holding. The wearable computing device may then generate a hologram corresponding to the medical device and overlays the hologram over the physical medical device in real time. Other examples are possible as well.

In such an example, the hologram corresponding to the three-dimensional model of the body part may comprise a first color, and the medical device hologram may comprise a second color that is different than the first color. Such a configuration may provide a distinction for the wearer between the hologram corresponding to the three-dimensional model of the body part and the medical device hologram so that movement of the medical device can be more clearly seen by the wearer.

In another example, a color of the hologram corresponding to the three-dimensional model of the body part may change based on the relative position of the medical device. For example, the color of the hologram corresponding to the three-dimensional model of the body part may change when the medical device is positioned in or contacts the physical body part. In another example, color of the hologram corresponding to the three-dimensional model of the body part may change as the medical device gets closer to the physical body part. For example, the color of the hologram corresponding to the three-dimensional model of the body part may be green when the medical device is a first distance from the physical body part, the color of the hologram may change to yellow as the medical device is a second distance from the physical body part that is closer than the first distance, and the color of the hologram may change to red once the medical device is positioned in or contacts the physical body part.

In another example, the wearable computing device provides haptic feedback based on the relative position of the medical device. For example, the wearable computing device may vibrate when the medical device is positioned in or contacts the physical body part. In another example, the wearable computing device may vibrate when the medical device is positioned incorrectly with respect to the target physical body part. In another example, the wearable computing device may vibrate differently as the medical device gets closer to the physical body part. For example, the wearable computing device may vibrate at a first frequency when the medical device is a first distance from the physical body part, the wearable computing device may vibrate at a second frequency as the medical device is a second distance from the physical body part that is closer than the first distance, and the wearable computing device may vibrate at a third frequency once the medical device is positioned in or contacts the physical body part. Other haptic feedback is possible as well.

In another example, the display may provide visual information based on the determined relative position of the medical device. For example, the visual information may include a display of a distance between the medical device and the target physical body part (i.e., x, y, and z coordinates). In another example, in a training example, the visual information may include a score after the procedure is complete. Such a score may be based on the accuracy of the movement of the medical device relative to the target physical body part. Other visual information is possible as well.

In another example, the display may provide one or more visual instructions configured to guide a wearer of the wearable computing device to perform a medical procedure with the medical device. In one example, the visual instructions include step by step instructions to perform the medical procedure. In another example, the visual instructions may include arrows, dotted lines, and/or other guiding visuals to help the wearer perform the medical procedure with the medical device.

In another example, the wearable computing device may include an audio output device. In such an example, the method may further include causing the audio output device to provide one or more audible instructions configured to guide a wearer of the wearable computing device to perform a medical procedure with the medical device. The one or more audible instructions may include step by step instructions to perform the medical procedure. In another example, the one or more audible instructions may include an indication that the medical device is positioned correctly with respect to the target physical body part. In another example, the one or more audible instructions may include an indication that the medical device is positioned incorrectly with respect to the target physical body part.

In another example, the one or more audible instructions may include beeping when the medical device is positioned in or contacts the physical body part. In another example, the wearable computing device may beep differently as the medical device gets closer to the physical body part. For example, the wearable computing device may beep at a first frequency when the medical device is a first distance from the physical body part, the wearable computing device may beep at a second frequency as the medical device is a second distance from the physical body part that is closer than the first distance, and the wearable computing device may beep at a third frequency once the medical device is positioned in or contacts the physical body part. Other audible instructions are possible as well.

In yet another example, the wearable computing device of the method 300 comprises a first wearable computing device. As such, the method 300 may be operable by both the first wearable computing device and a second wearable computing device. In such an example, the method 300 may further include receiving, by the second wearable computing device, the data indicative of the three-dimensional model of the body part. The method 300 may further include receiving sensor data from one or more sensors of the second wearable computing device. The method 300 may further include, based on the sensor data, determining a relative position of the physical body part of the physical body with respect to the second wearable computing device. The method 300 may further include, based on the determined relative position, causing a partially or fully transparent display of the second wearable computing device to provide a hologram corresponding to at least a portion of the three-dimensional model of the body part such that the hologram appears overlaid onto at least a portion of the physical body part when viewed through the display of the second wearable computing device. The method 300 may further include determining, by the second wearable computing device, a relative position of a medical device with respect to the second wearable computing device. In such an example, the method 300 may further include, based on the determined relative position of the medical device, the second wearable computing device causing the display of the second wearable computing device to provide a medical device hologram corresponding to at least a portion of the medical device that is positioned in the physical body.

As such, multiple wearers can perceive holograms overlaid on top of the same physical body part of the patient and further perceive holograms overload on top of the same medical device. Such an arrangement may be useful in medical procedures where more than one medical professional are required to perform the procedure. Such an arrangement may also be useful in training exercises, such that a trainee could observe an instructor conducting a medical procedure and/or such that an instructor could supervise a trainee during a practice procedure. Other uses are possible as well.

### D. ILLUSTRATIVE WEARABLE COMPUTING DEVICE FUNCTIONALITY

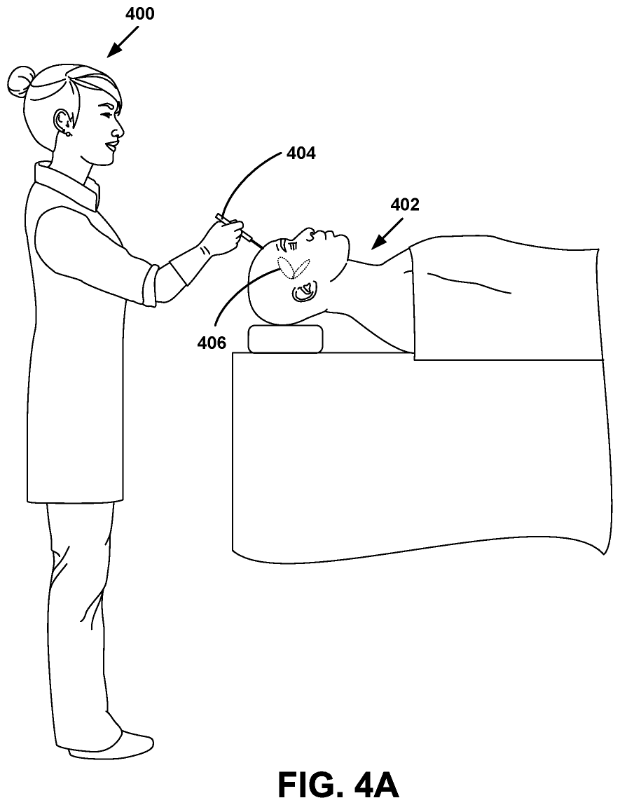

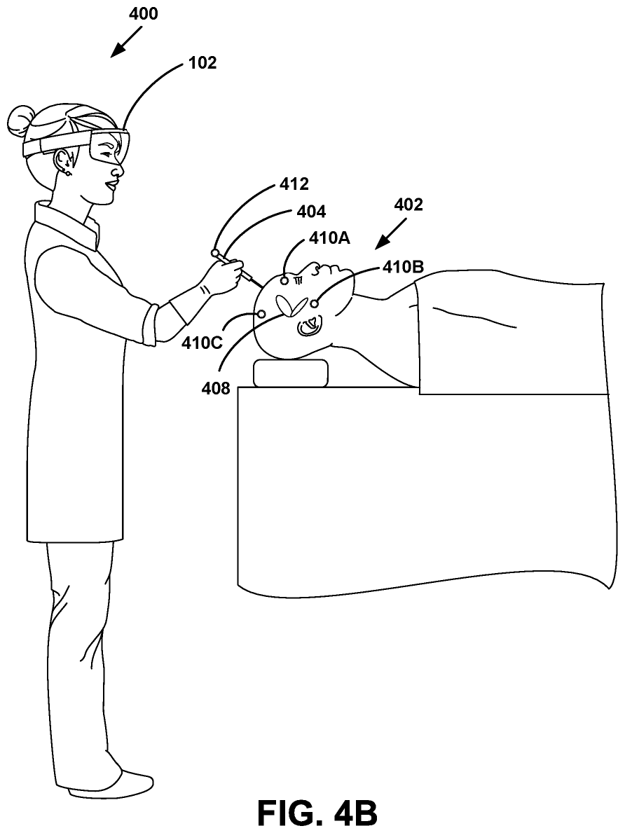

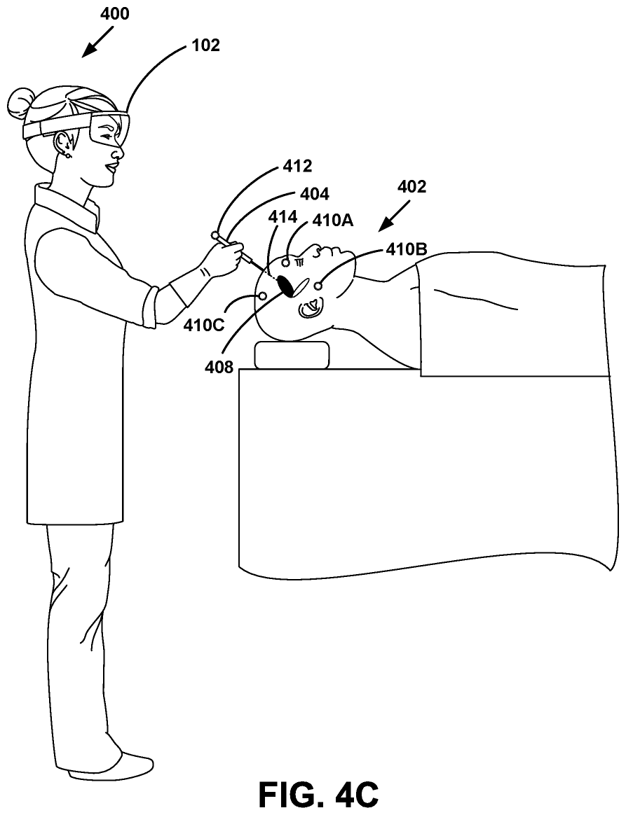

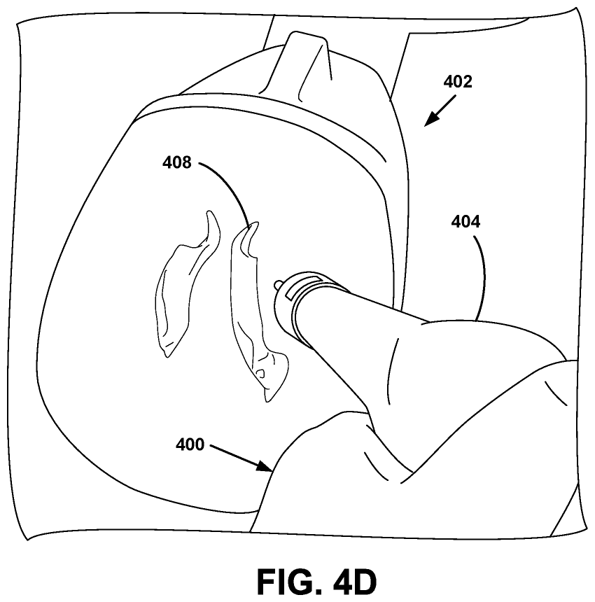

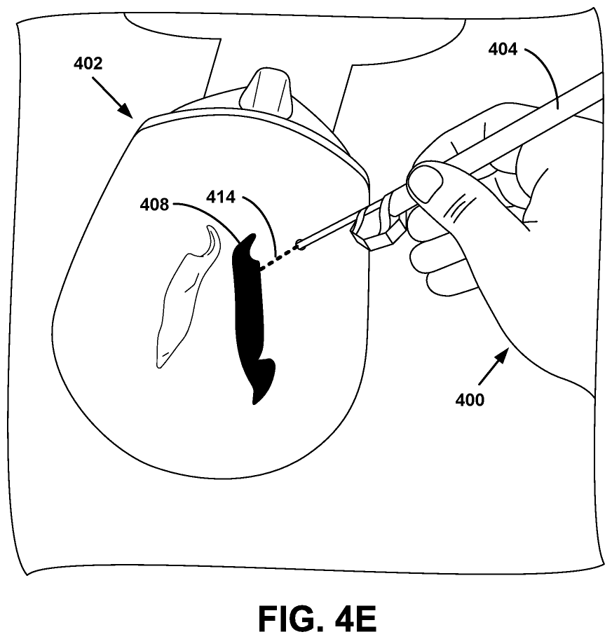

FIGS. 4A, 4B 4C, 4D, and 4E illustrate applications of the wearable computing device as described above, according to example embodiments. In order to provide the various functionalities described herein, the wearable computing device may utilize methods such as those described above in reference to FIG. 3. However, other techniques may also be used to provide the wearable computing device functionality shown in FIGS. 4A, 4B 4C, 4D, and 4E.

FIG. 4A illustrates a medical professional 400 performing a medical procedure, according to an example embodiment. As shown in FIG. 4A, the medical professional 400 is performing a medical procedure on a physical body 402 without the use of a wearable computing device. In particular, the medical professional 400 is attempting to position a medical device 404 (a probe in this particular implementation) into a physical body part 406 (ventricle of the brain in this particular implementation) of the physical body 402. However, the physical body part 406 in this case is hidden from view. Such a procedure may be challenging to perform since the medical professional 400 cannot see the physical body part 406 upon which they are performing the procedure.

FIG. 4B illustrates a medical professional 400 with a wearable computing device (such as the wearable computing device 102 that is described above with reference to FIG. 1) performing the medical procedure shown in FIG. 4A, according to an example embodiment. In particular, FIG. 4B illustrates a hologram 408 of a three-dimensional model of a body part such that the hologram 408 appears overlaid onto the physical body part 406 when viewed through the display of the wearable computing device 102. As discussed above, in one example the hologram 408 of the three-dimensional model of the body part may be overlaid onto at least a portion of the physical body part 406 by aligning three or more markers 410A-410C positioned on the physical body 402 (e.g., via a temporary adhesive on one side of each of the three or more markers). The physical body part 406 of the physical body 402 may then be captured using an image capture system, such as a CT scan. The CT scan captures both the physical body part 406, and the three or more markers 410A-410C positioned on the physical body 402. The distance between the three or more markers 410A-410C and the physical body part 406 may then be used to determine the relative position of the physical body part 406 of the physical body 402 with respect to the wearable computing device 102, and may further be used to align the hologram 408 of the three-dimensional model of the body part such that the hologram 408 appears overlaid onto the physical body part 406 when viewed through the display of the wearable computing device 102. Other alignment steps are possible as well.

As discussed above, the wearable computing device 102 may determine a relative position of a medical device 404 with respect to the wearable computing device 102. The relative position of the medical device 404 may be determined in number of ways. In one example, the medical device 404 may include a tracking sensor 412 that the wearable computing device 102 can detect to determine the location of the medical device 404 relative to the wearable computing device 102. In another example, a plurality of markers may be positioned on the medical device 404 that the wearable computing device 102 can detect to determine the location of the medical device 404 relative to the wearable computing device 102. In yet another example, the wearable computing device 102 may store or have access to holographic models of a plurality of medical devices. In such an example, the medical professional 400 then selects via the wearable computing device 102 the model of the medical device that matches the physical one they are holding. The wearable computing device 102 may then generate a hologram corresponding to the medical device 404 and overlays the hologram over the physical medical device in real time. Other examples are possible as well.

FIG. 4C illustrates the medical professional 400 of FIG. 4B further performing the medical procedure, according to an example embodiment. As shown in FIG. 4C, the wearable computing device 102 may display a medical device hologram 414 corresponding to (e.g., registered to or otherwise aligned with) at least a portion of the medical device 404 that is positioned in the physical body 402. In an example embodiment, the medical device hologram 414 may provide a virtual representation of the portion of the medical device 404 positioned within the physical body 402, which would normally be obscured from direct viewing. As such, the hologram 414 of the portion of the medical device 404 that is positioned in the physical body 402 becomes visible to the wearer once that portion is positioned in the physical body 402 and hidden from view from someone who is not wearing the wearable computing device 102. As shown in FIG. 4C, the color of the hologram 408 of the three-dimensional model of the body part may change based on the relative position of the medical device 404. For example, as shown in FIG. 4C, the color of the hologram 408 of the three-dimensional model of the body part may change when the medical device 404 is positioned in or contacts the physical body part 406.

FIG. 4D illustrates a first person view of the medical professional 400 of FIG. 4B performing the medical procedure of FIG. 4B, according to an example embodiment. In particular, FIG. 4D illustrates a hologram 408 of a three-dimensional model of a body part such that the hologram 408 appears overlaid onto the physical body part 406 when viewed through the display of the wearable computing device 102. FIG. 4E illustrates a first person view of the medical professional 400 of FIG. 4B performing the procedure of FIG. 4C, according to an example embodiment. In particular. FIG. 4E illustrates how the wearable computing device 102 could display a medical device hologram 414 corresponding to at least a portion of the medical device 404 that is positioned in the physical body 402. As such, the hologram 414 of the portion of the medical device 404 that is positioned in the physical body 402 becomes visible to the wearer once that portion is positioned in the physical body 402 and hidden from view from someone who is not wearing the wearable computing device 102.

Although the medical procedure illustrated in FIGS. 4A-4E relates to the ventricles of the brain, the methods and functionality described herein relate to many other medical procedures, including training procedures. For example, within neurosurgery other potential uses are placement of deep brain stimulator electrodes, placement of hardware, such as screws, rods, catheters, or electrodes into the spine, navigating to and operating on a brain tumor or vascular abnormality (aneurysm, AVM, fistula), placement of SEPS drain, placement of a reservoir for CSF sampling or introduction of pharmaceuticals, placement of intracranial pressure monitor or LiCOX system, endoscopic nasal procedures, spinal cord stimulators, radiofrequency ablations. Outside of neurosurgery there are also many additional possible uses, including placement of central lines, percutaneous biopsies of liver, lung, skin, thyroid, kidney, percutaneous drain placement for abscess, fluid collections, CT or ultrasound guided procedures, chest tube placement. Placement of a catheter, electrode, drain, or medical device into a body organ or space by real-time augmented reality navigation. Other medical procedures are possible as well.

### E. EXAMPLE COMPUTER-READABLE MEDIUM

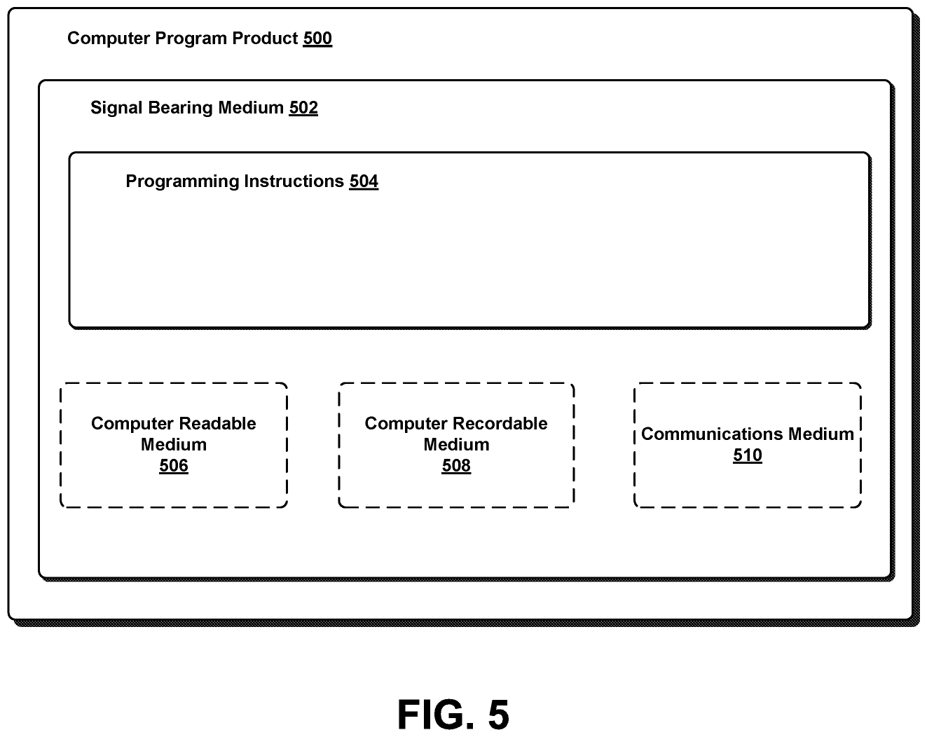

FIG. 5 illustrates a computer-readable medium configured according to an example embodiment. In example embodiments, the example system can include one or more processors, one or more forms of memory, one or more input devices/interfaces, one or more output devices/interfaces, and machine-readable instructions that when executed by the one or more processors cause the system to carry out the various functions, tasks, capabilities, etc., described above.

As noted above, in some embodiments, the disclosed methods can be implemented by computer program instructions encoded on a non-transitory computer-readable storage media in a machine-readable format, or on other non-transitory media or articles of manufacture. FIG. 5 is a schematic illustrating a conceptual partial view of an example computer program product that includes a computer program for executing a computer process on a computing device, arranged according to at least some embodiments presented herein.

In one embodiment, the example computer program product 500 is provided using a signal bearing medium 502. The signal bearing medium 502 may include one or more programming instructions 504 that, when executed by one or more processors may provide functionality or portions of the functionality described above with respect to FIGS. 1-4C. In some examples, the signal bearing medium 502 can be a computer-readable medium 506, such as, but not limited to, a hard disk drive, a Compact Disc (CD), a Digital Video Disk (DVD), a digital tape, memory, etc. In some implementations, the signal bearing medium 502 can be a computer recordable medium 508, such as, but not limited to, memory, read/write (R/W) CDs, R/W DVDs, etc. In some implementations, the signal bearing medium 502 can be a communications medium 510, such as, but not limited to, a digital and/or an analog communication medium (e.g., a fiber optic cable, a waveguide, a wired communications link, a wireless communication link, etc.). Thus, for example, the signal bearing medium 502 can be conveyed by a wireless form of the communications medium 510.

The one or more programming instructions 504 can be, for example, computer executable and/or logic implemented instructions. In some examples, a computing device such as the processor 214 of FIG. 2 is configured to provide various operations, functions, or actions in response to the programming instructions 504 conveyed to the processor 214 by one or more of the computer-readable medium 506, the computer recordable medium 508, and/or the communications medium 510.

The non-transitory computer-readable medium could also be distributed among multiple data storage elements, which could be remotely located from each other. The device that executes some or all of the stored instructions could be a client-side computing device 210 as illustrated in FIG. 2. Alternatively, the device that executes some or all of the stored instructions could be a server-side computing device.

### F. CONCLUSION

The above detailed description describes various features and functions of the disclosed systems, devices, and methods with reference to the accompanying Figures. In the Figures, similar symbols typically identify similar components, unless context dictates otherwise. The illustrative embodiments described in the detailed description, Figures, and claims are not meant to be limiting. Other embodiments can be utilized, and other changes can be made, without departing from the scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

1. A wearable computing device comprising:
one or more sensors;
a partially or fully transparent display; and
a control system configured to:
receive data indicative of a three-dimensional model of a body part;
receive sensor data from the one or more sensors;
based on the sensor data, determine a relative position of a physical body part of a physical body with respect to the wearable computing device; and
based on the determined relative position, cause the display to provide a hologram corresponding to at least a portion of the three-dimensional model of the body part such that the hologram appears overlaid onto at least a portion of the physical body part when viewed through the display.

2. The wearable computing device of claim 1, wherein the wearable computing device is implemented as part of or takes the form of a head-mountable device (HMD).

3. The wearable computing device of of claim 1, wherein the one or more sensors comprise one or more of: (a) one or more proximity sensors, (b) one or more touch interfaces, (c) one or more microphones, (d) one or more accelerometers, (e) one or more gyroscopes, or (f) one or more magnetometers.

4. The wearable computing device of claim 1, wherein the control system is further configured to:
determine a relative position of a medical device with respect to the wearable computing device; and
based on the determined relative position of the medical device, cause the display to provide a medical device hologram corresponding to at least a portion of the medical device that is positioned in the physical body.

5. The wearable computing device of claim 4, wherein the hologram corresponding to the three-dimensional model of the body part comprises a first color, and wherein the medical device hologram comprises a second color that is different than the first color.

6. The wearable computing device of claim 4, wherein a color of the hologram corresponding to the three-dimensional model of the body part changes based on the relative position of the medical device.

7. The wearable computing device of claim 4, wherein the control system is further configured to:
cause the display to provide visual information based on the determined relative position of the medical device.

8. The wearable computing device of claim 4, wherein the wearable computing device provides haptic feedback based on the relative position of the medical device.

9. The wearable computing device of claim 4, wherein the control system is further configured to:
cause the display to provide one or more visual instructions configured to guide a wearer of the wearable computing device to perform a medical procedure with the medical device.

10. The wearable computing device of claim 4, further comprising an audio output device, wherein the control system is further configured to:
cause the audio output device to provide one or more audible instructions configured to guide a wearer of the wearable computing device to perform a medical procedure with the medical device.

11. The wearable computing device of claim 1, further comprising an audio input device, wherein the control system is further configured to:
receive, from the audio input device, one or more verbal inputs; and
adjust a location of the hologram based on the one or more verbal inputs.

12. The wearable computing device of claim 1, wherein the data indicative of the three-dimensional model of the body part is based on a computed tomography (CT) scan of a brain.

13. The wearable computing device of claim 1, wherein the data indicative of the three-dimensional model of the body part comprises a real-time three-dimensional model of the physical body part.

14. The wearable computing device of claim 1, wherein determining the relative position of the physical body part of the physical body with respect to the wearable computing device is further based on three or more markers positioned on the physical body.

15. A computer-implemented method operable by a wearable computing device, the method comprising:
receiving data indicative of a three-dimensional model of a body part;
receiving sensor data from one or more sensors of the wearable computing device;
based on the sensor data, determining a relative position of a physical body part of a physical body with respect to the wearable computing device; and
based on the determined relative position, causing a partially or fully transparent display of the wearable computing device to provide a hologram corresponding to at least a portion of the three-dimensional model of the body part such that the hologram appears overlaid onto at least a portion of the physical body part when viewed through the display.

16. The method of claim 15, further comprising:
determining, by the wearable computing device, a relative position of a medical device with respect to the wearable computing device; and
based on the determined relative position of the medical device, causing the display to provide a medical device hologram corresponding to at least a portion of the medical device that is positioned in the physical body.

17. The method of claim 15, wherein the wearable computing device comprises a first wearable computing device, and wherein the method is operable by both the first wearable computing device and a second wearable computing device, the method further comprising:
receiving, by the second wearable computing device, data indicative of the three-dimensional model of the body part;
receiving, by the second wearable computing device, sensor data from one or more sensors of the second wearable computing device;
based on the sensor data, the second wearable computing device determining a relative position of the physical body part of the physical body with respect to the second wearable computing device; and
based on the determined relative position, the second wearable computing device causing a partially or fully transparent display of the second wearable computing device to provide a hologram corresponding to at least a portion of the three-dimensional model of the body part such that the hologram appears overlaid onto at least a portion of the physical body part when viewed through the display of the second wearable computing device.

18. The method of claim 17, further comprising:
determining, by the second wearable computing device, a relative position of a medical device with respect to the second wearable computing device; and
based on the determined relative position of the medical device, the second wearable computing device causing the display of the second wearable computing device to provide a medical device hologram corresponding to at least a portion of the medical device that is positioned in the physical body.

19. A non-transitory computer-readable medium having stored thereon instructions that, when executed by one or more processors of a wearable computing device, cause the wearable computing device to perform functions comprising:
receiving data indicative of a three-dimensional model of a body part;
receiving sensor data from one or more sensors of the wearable computing device;
based on the sensor data, determining a relative position of a physical body part of a physical body with respect to the wearable computing device; and
based on the determined relative position, causing a partially or fully transparent display of the wearable computing device to provide a hologram corresponding to at least a portion of the three-dimensional model of the body part such that the hologram appears overlaid onto at least a portion of the physical body part when viewed through the display.

20. The computer-readable medium of claim 19, wherein the functions further comprise:
determining a relative position of a medical device with respect to the wearable computing device; and
based on the determined relative position of the medical device, causing the display to provide a medical device hologram corresponding to at least a portion of the medical device that is positioned in the physical body.
